(19) United States
(12) Patent Application Publication
(10) Pub. No.: US 2022/0313211 A1
(43) Pub. Date: Oct. 6, 2022

(54) ULTRASOUND SUPPORT DEVICE

(71) Applicants: Mahmoud I. Eltorai, Marlborough, MA (US); Adam E. M. Eltorai, Marlborough, MA (US)

(72) Inventors: Mahmoud I. Eltorai, Marlborough, MA (US); Adam E. M. Eltorai, Marlborough, MA (US)

(21) Appl. No.: 17/712,733

(22) Filed: Apr. 4, 2022

Related U.S. Application Data
(60) Provisional application No. 63/170,724, filed on Apr. 5, 2021.

(51) Int. Cl. A61B 8/00; A61B 50/00; F16M 13/02

(52) U.S. Cl. CPC A61B 8/4218; A61B 8/462; A61B 50/00; F16M 13/022

(57) ABSTRACT

A medical probe support device provides one or more support arms and corresponding receptacles for receiving medical imaging components, such as scanners, rendering screens and needles, and supports the components in a fixed but adjustable position to free the hands of the scan operator or technician during the scan. Each of the support arms has a balance of rigidity and resilience to support the imaging components in a set position and allow repositioning of the imaging components by exerting a slight repositioning force to overcome the rigidity. The support arm is an elongated, flexible member with a proximate end attached to the imaging component and a distal end secured to a fixture. The proximate end is responsive to temporary repositioning during a scan procedure, and the receptacle may have an adjacent needle guide for inserting a needle relative to the scan device for monitoring needle advancement.

### RELATED APPLICATIONS

This patent application claims the benefit under 35 U.S.C. § 119(e) of U.S. Provisional Patent App. No. 63/170,724, filed Apr. 5, 2021 entitled “ULTRASOUND PROBE SUPPORT DEVICE,” incorporated herein by reference in entirety.

### BACKGROUND

Scanning technology has driven substantial advances for medical diagnostics by providing visual assessment of internal anatomical structures without surgical intervention. Imaging technology such as Computed Axial Tomography (CAT, or simply CT), Magnetic Resonance Imaging (MRI), and Ultrasonic or Ultrasound (US) are all employed in various contexts for scanning internal anatomical structures. Ultrasound imaging is particularly beneficial because it can be implemented in a hand-held probe that is easily maneuverable over an examination region. Hand-held, portable US probe imaging also allows the operator to perform image-guided diagnostic or therapeutic procedures for patients such as needle biopsies, fluid aspiration, injections and vascular access, to name several.

### SUMMARY

A medical probe support device provides one or more support arms and corresponding receptacles for receiving medical imaging components, such as scanners, rendering screens and needles, and supports the components in a fixed but adjustable position to free the hands of the scan operator or technician during the scan. Each of the support arms has a balance of rigidity and resilience to support the imaging components in a set position and allow repositioning of the imaging components by exerting a slight repositioning force to overcome the rigidity. The support arm is an elongated, flexible member with a proximate end attached to the imaging component and a distal end secured to a fixture. The proximate end is responsive to temporary repositioning during a scan procedure, and the receptacle may have an adjacent needle guide for inserting a needle relative to the scan device for monitoring needle advancement. In this manner, a scan procedure may invoke a rendering screen positioned at a vantage point for observation of the imaged region as the scan operator positions the scan device, and then advances the needle while the monitor and scan remain positioned by respective support arms.

Configurations herein are based, in part, on the observation that the US probes are often deployed as a single-user task in a patient room. Since the handheld probe is freely tethered, unlike large MRI patient tables, the scan operator typically manipulate the US probe with one hand while performing patient manipulations and device controls with their other hand. For example, the technician may need to apply patient pressure for positioning scanned anatomical structures, or may need to manipulate scan parameters such as signal strength, image contrast and the like. In addition, the hand-held US probe allows the operator to perform image-guided diagnostic or therapeutic procedures for patients such as needle biopsies, fluid aspiration, injections and vascular access. Accordingly, configurations herein substantially overcome the shortcomings of conventional approaches by providing a receptacle and support arm for free positioning of the US probe adjacent the examination region, effectively freeing up both hands of the operator to perform the image-guided interventions.

### BRIEF DESCRIPTION OF THE DRAWINGS

The foregoing and other objects, features and advantages of the invention will be apparent from the following description of particular embodiments of the invention, as illustrated in the accompanying drawings in which like reference characters refer to the same parts throughout the different views. The drawings are not necessarily to scale, emphasis instead being placed upon illustrating the principles of the invention.

FIG. 1 is a perspective view of a particular configuration of a support device for ultrasound probes and similar handheld medical scanning devices;

FIGS. 1A and 1B show an example of a receptacle engaging a probe or image display as in FIG. 1;

FIG. 2 is an alternate configuration for attachment to an IV pole or bedside rail;

FIGS. 3A and 3B include an alternate configuration having opposed hinged annular jaws;

FIG. 4, depicts a configuration including fixed annular clamp has annular protrusions;

FIGS. 5A-5C show a schematic view of a probe and rendering screen configuration of the support device of FIG. 1;

FIG. 6 shows a functional block diagram of the dual probe and display screen configuration of FIGS. 5A-5C; and

FIG. 7 shows a side view of a deployed and communicative probe and display screen configuration as in FIG. 6.

### DETAILED DESCRIPTION

Configurations below describe implementation of an ultrasound probe in conjunction with a monitor and optionally a needle guide. A hand US imager and a smartphone sized rendering screen (imaging screen) are shown as examples, however any suitable handheld probe and corresponding monitor device may be employed, based on the rigidity and inertial resilience and stiffness of the support arm. A single support arm may be employed, and additional support arms extending from a common base or attachment secures the distal end of a plurality of support arms for respective imaging components. US probes are an example for providing quick, inexpensive imaging of a patient, however the imaging medium and technology in the supported probe may vary.

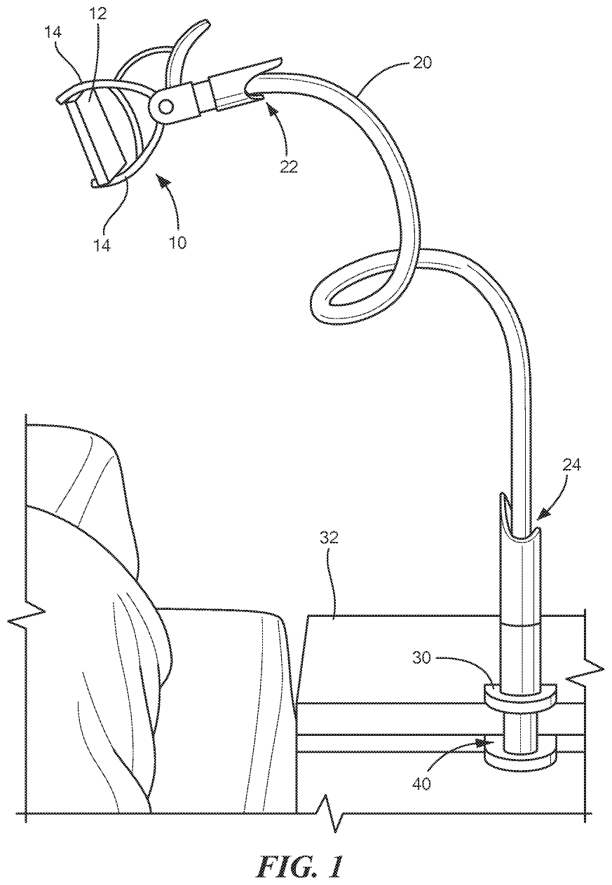

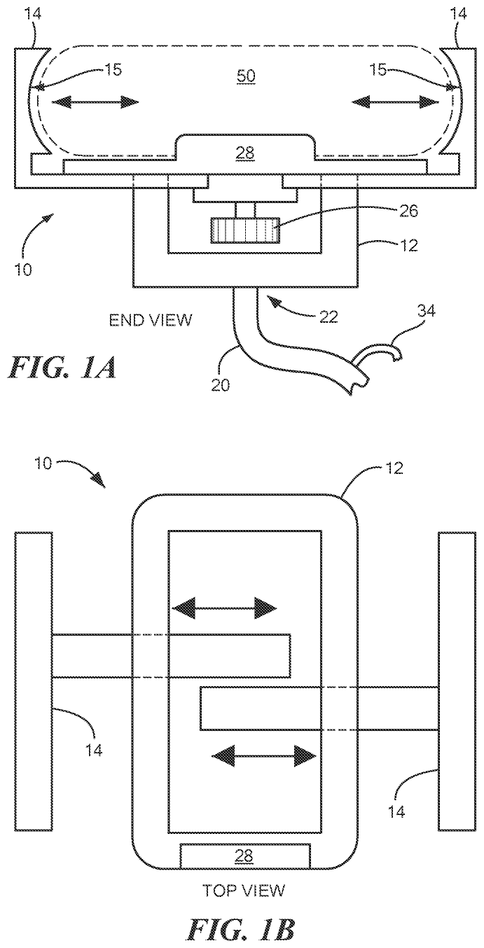

FIG. 1 is a perspective view of a particular configuration of a support device for ultrasound probes and similar handheld medical scanning devices. Referring to FIG. 1, in a general arrangement, a brace device for a medical imaging probe such as an US probe includes a clamp, holder or receptacle 10 for engaging an imaging probe. FIG. 1 sets out a general arrangement of the elements. The receptacle 10 may include a frame 12 or similar structure having opposed lateral braces 14 for compressive, frictional or rigid engagement with the imaging probe. A support arm 20 has a proximate end 22 and a distal end 24, and the support arm 20 attaches to the frame 12 at the proximate end and is adapted to support the imaging probe in a temporary fixed position. An attachment coupling 30 at the distal end 24 of the support arm, is adapted to engage a fixed support 32 or fixture, such as a cart, IV post or bedrail for bearing a combined weight of the imaging probe, receptacle and support arm. The support arm 20 has a spring-like resiliency for responsiveness to repositioning force and a rigidity for maintaining a repositioned orientation based on the repositioning force, such that the doctor or medical technician can readily reposition the probe in different positions around the examination region, and the support arm maintains the repositioned location of the receptacle 10 and engaged component.

US probes are beneficial because they are generally more agile than CT and MRI counterparts. US devices can be configured on a rollable cart for portability, rolled into a patient room, and the relatively small size of the probe lends it to handheld usage or receptacle assisted positioning as disclosed herein. The ultrasonic medium has properties that allow safe imaging of an unborn fetus, need not be operated in an absence of metal, and does not require shielding as conventional X-ray imaging requires.

FIGS. 1A and 1B show an example of a receptacle 10 as in FIG. 1. Referring to FIGS. 1-1B, a brace device for a medical imaging probe as in FIG. 1 includes the receptacle 10 for engaging an imaging component 50. The receptacle 10 includes a frame 12 having opposed lateral braces 14 for compressive engagement with the engaged component 50, such as a probe or image display (i.e. screen). the opposed lateral braces 14 on the frame 12 define a concave recession 15 for frictional engagement with a longitudinal side of the imaging component 50. A contraction control 26 on the frame is configured to adjust a distance between the opposed lateral braces 14 for accommodating the imaging probe in a frictional engagement as the braces 14 expand and contract to grip various sizes of components 50. An end brace 28 may extend from the frame in a perpendicular orientation to the opposed lateral braces 14, to prevent the probe from sliding out from between the braces 14. The end brace 28 is adapted to engage a width of the component 50 on a side of the imaging probe substantially perpendicular to the longitudinal side, typically the width of the probe for a substantially rectangular or oval shaped probe design.

The support arm 20 attaches to the frame 12 at the proximate end and is adapted to support the imaging probe in a temporary fixed position. An attachment fixture 30 at the distal end 24 of the support arm, is adapted to engage a fixed support 32 such as a cart, IV post or bedrail for bearing a combined weight of the imaging probe, cradle and support arm. The support arm 20 has a spring-like resiliency for responsiveness to repositioning force and a rigidity for maintaining a repositioned orientation based on the repositioning force, such that the doctor or medical technician can readily reposition the probe in different positions around the examination region. Other suitable engagements between the proximate end 22 of the support arm 20 and a respective imaging component 50 may be utilized. The receptacle 10 may engage the component 50 in any suitable manner, such as circumferentially, compressively as shown, tethered, i.e. strapped to a base, or other suitable engagement.

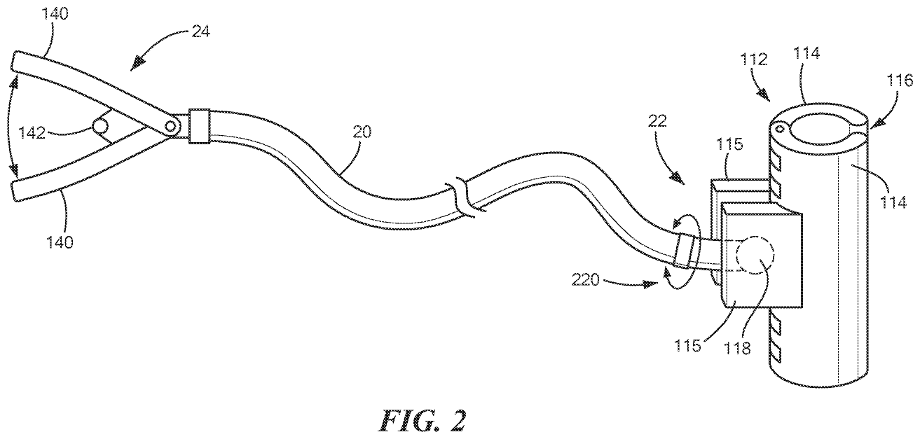

FIG. 2 is an alternate configuration for attachment to an IV pole or bedside rail. Referring to FIGS. 1 and 2, the support arm 20 has an elongated, deformable shaft for holding a deformed position against the weight of the receptacle and imaging probe, and deforms in response to forces greater than the weight of the receptacle and imaging probe. A rotating pivot or ball and socket joint attaches the proximate end 22 to the receptacle 10. The support arm may be a continuous spring-like metal, coil or polymer material, or a combination thereof, that has a combination of flexibility and rigidity to support the weight of the probe, but bend when manipulated by the scan technician. The support arm 20 may also include multiple rigid sections attached by articulating, hinged or 360 degree pivot joints. The support 20 arm may include one or more clasps for securing an electric cable in a substantially adjacent arrangement with the support arm, typically required for communication and power with the imaging control resting on an adjacent cart. Alternatively, a wireless medium may be employed between the probe and the imaging control.

In a particular configuration, shown in FIG. 2, the proximate end 22 employs a circular holder 112 employing opposed annular jaws 114 defining a groove 116 or receptacle for engaging a probe. Tabs 115 allow spring biased closure of the opposed jaws 114, biased by spring 118. The jaw 114 and tab 115 engage the support arm 20 via a rotatable linkage 220. Any of the disclosed jaw assemblies may employ a rotatable attachment for 360 degree movement. The distal end 24 has opposed straight jaws 140 biased by a spring 142 for clamping or closure around a fixture.

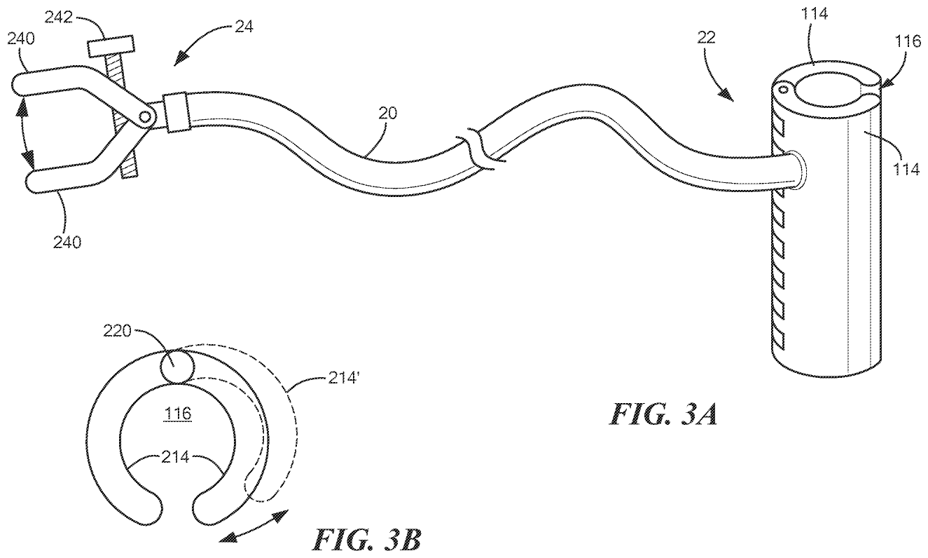

FIGS. 3A and 3B include an alternate configuration having opposed hinged annular jaws 214 defining the groove 116. Referring to FIGS. 1-3, a hinge 220 allows opening of the jaw 214′. A suitable spring or clamp engagement closes the jaws 214 around a probe disposed in the groove 116 region. At the distal end 24, opposed angled jaws 240 allow closure around a fixture through a rotating threaded clamp 242 mechanism.

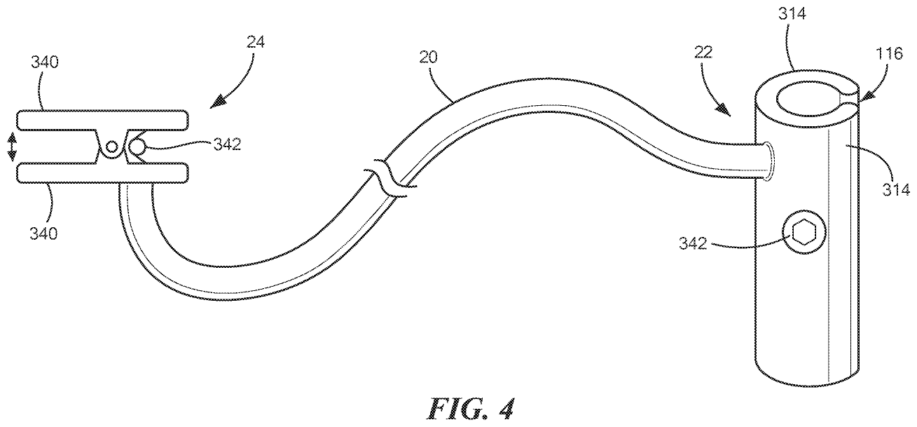

FIG. 4, depicts a configuration including fixed annular clamp. In FIG. 4, the fixed annular clamp has annular protrusions 314 at the proximate end 22, and a rotating threaded member 342 extends into the groove 116 region for a frictional interference fit with a fixture such as a solid bar. At the distal end, straight pivoting jaws 340 are biased by spring 342 for closure around a suitable fixture.

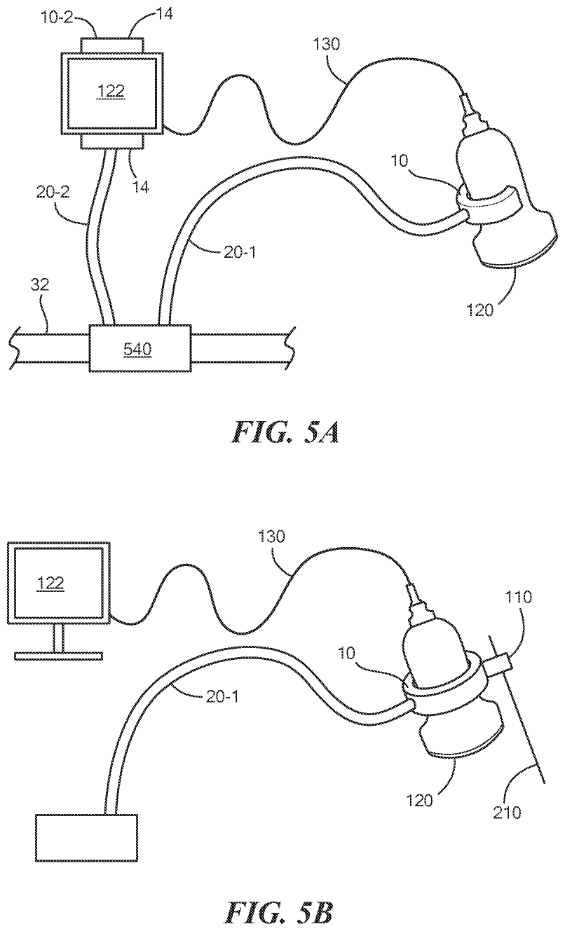

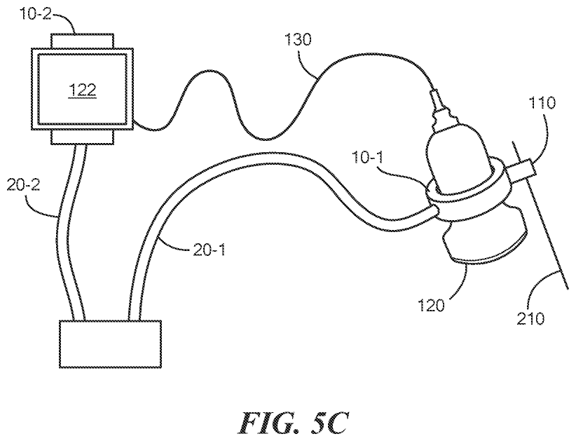

FIGS. 5A-5C show a schematic view of a probe and rendering screen configuration of the support device of FIG. 1. Referring to FIGS. 1-5C, a plurality of support arms 20-1 . . . 20-2 (20 generally) may be employed to support a probe 120 and an imaging display 122. FIG. 5A shows an approach as in FIG. 1 that also employs a second support arm 20-2 having a second receptacle 10-2 for holding the probe 120 or imaging display 122. The receptacle 10-2 engaging the image display 122 may likewise employ lateral braces 14 or other suitable engagement. Additional support arms 20 may be employed, however may not be needed as an excessive number of imaging components may suggest a need for additional operators.

The receptacle 10 may include a frame and 12 and opposing lateral braces 14 as in FIG. 1, or may engage the probe 120 in an alternate manner, often specific to the shape of the probe. The receptacle 10 need only maintain a detachable engagement with the probe, and may include frictional, interference or tethered (e.g. straps) for engaging the probe 120 with the receptacle. A signal cable 130 transmits a video signal from the probe 120 to the image display 122. Both the image display 122 and the probe 120 remain secured by respective support arms 20-N from a common base 540 secured to a fixture 32 or other fixed support.

In FIG. 5B, the probe receptacle 10 has a needle guide 110, which secures a needle 210 to the receptacle 10. This maintains the needle 210 and probe 120 in a relative orientation. In an expected scenario, the probe operator first positions the probe 120 by positioning the receptacle 10, and then manipulates the needle 210 while viewing the needle path on the image display 122.

FIG. 5C combines dual support arms 20-1 . . . 20-2 with the needle guide 110 on the probe receptacle 10-1. This provides flexibility in positioning the image screen 122 in a good line of sight once positioning the probe 120.

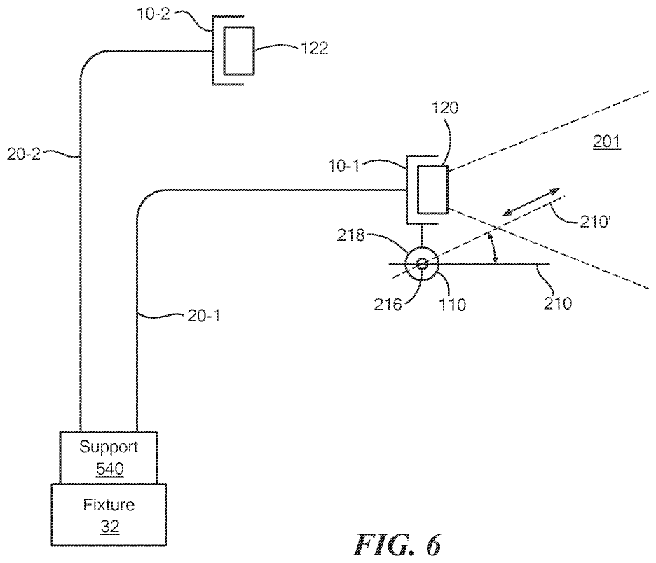

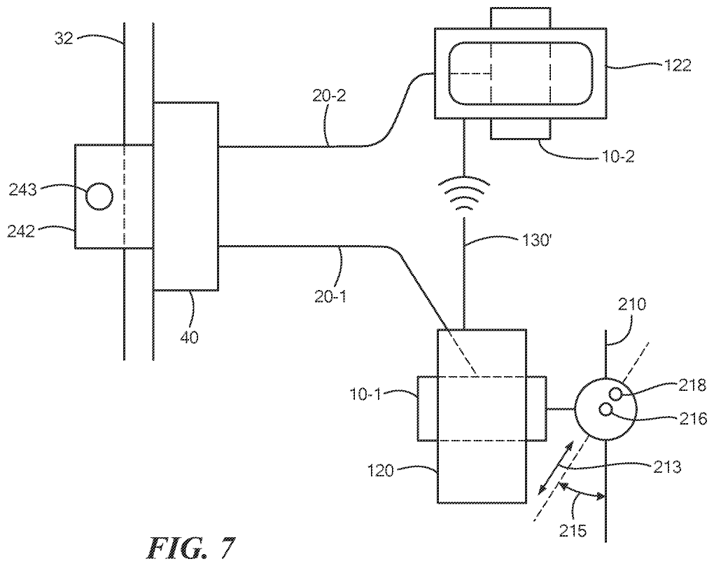

FIG. 6 shows a functional block diagram of the dual probe and display screen configuration of FIGS. 5A-5C. In FIG. 6, the needle guide 110 is disposed adjacent to the probe receptacle 10-1 for directing a needle 210 in a direction of a probed region 201 based on an orientation of the probe 120 disposed in the receptacle. The needle guide 110 has a translation control 218 for advancing and retracting the needle in the direction of the probed region. The needle guide 110 also has a trajectory angle control 216. The trajectory angle control 216 having a selectable stop for securing the needle at a trajectory angle for intersecting the probed region upon forward translation of the needle in response to the translation control. In other words, the needle guide 110 allows the needle to be angled and inserted towards a probed surgical target by viewing the advancing needle on the image screen 122.

FIG. 7 shows a side view of a deployed and communicative probe and display screen configuration as in FIG. 6. In FIG. 7, the attachment coupling 40 has a jaw adapted for engaging a tubular or nontubular fixture 32 in a compressive arrangement. A threaded clamp 242 employs a rotatable control 243 or other spring biased, frictional, compressive or interference based engagement with a stable fixture 32 supportive of the base 40, support arms 20, and engaged components.

Respective receptacle 10-1, 10-2 support the probe 120 and associated imaging display may also be connected by a wireless connection 130′. Other components include the needle guide 110. The needle guide may define a rotatable and translatable engagement with the needle 210, such as by threaded controls 212, 214. The translation control is a threaded member allowing forward insertion and retraction of the needle 210, as shown by arrow 213. The trajectory angle control 216 includes a rotatable disl fixed by a threaded member allowing rotation or pivot control, as shown by arrow 215. Other suitable needle insertion and guidance capabilities may be engaged with the receptacle 10-1. An advantage of fixing the needle guide 110 to the receptacle 10-1 is binding both the probe 120 and the needle 210 to a common plane of reference for accuracy of imaging while directing the needle towards a surgical target. Independent positioning of the display image 122 allows repositioning of the display image 122 for a direct visual path by the needle operator once positioning the probe/needle receptacle 10-1 in an optimal position.

While the system and methods defined herein have been particularly shown and described with references to embodiments thereof, it will be understood by those skilled in the art that various changes in form and details may be made therein without departing from the scope of the invention encompassed by the appended claims.

What is claimed is:

1. A device for securing an ultrasound probe and/or an ultrasound image display in an optimal position to free the hands of the operator for percutaneous ultrasound-guided procedures.

2. The device of claim 1, further comprising:
a receptacle for engaging the probe or image display;
a support arm having a proximal end and a distal end, the support arm attached to the receptacle at the proximal end and adapted to support the imaging probe or image display in a temporary fixed position and the support arm having a resiliency for responsiveness to repositioning force and a rigidity for maintaining a repositioned orientation based on the repositioning force; and
an attachment coupling at the distal end of the support arm, the attachment coupling adapted to engage a fixed support for bearing a combined weight of the imaging probe or image display, receptacle and support arm.

3. The device of claim 2, further comprising a second support arm having a second receptacle for holding the probe or image display.

4. The device of claim 2, wherein the receptacle defines a concave recession for frictional engagement with a longitudinal side of the imaging probe and/or image display.

5. The device of claim 2, wherein the receptacle is adjustable to fit different size ultrasound probes and/or image displays.

6. The device of claim 2, wherein the receptacle has a detachable engagement with the proximal end of the support arm for exchanging different receptacles.

7. The device of claim 2, wherein at least one component formed from material responsive to cleaning and/or sterilization.

8. The device of claim 2, wherein the receptacle is defined by opposed, retractable claws, jaws, or braces adapted for compression biasing around a probe and/or image display, the opposed, retractable claws, jaws, or braces attached to the proximal end of the support arm by a rotatable attachment.

9. The device of claim 2, wherein the support arm has a deformable shaft, the deformable shaft for holding a deformed position against the weight of the receptacle and imaging probe and/or image display, and deforms in response to forces greater than the weight of the receptacle and imaging probe and/or image display.

10. The device of claim 2, wherein the receptacle has a needle guide, the needle guide disposed adjacent to the receptacle for directing a needle in a direction of a probed region based on an orientation of a probe disposed in the receptacle.

11. The device of claim 10, wherein the needle guide has a translation control, the translation control for advancing and retracting the needle in the direction of the probed region.

12. The device of claim 11, wherein the needle guide has a trajectory angle control, the trajectory angle control having a selectable stop, the selectable stop for securing the needle at a trajectory angle for intersecting the probed region upon forward translation of the needle in response to the translation control.

13. The device of claim 2, wherein the attachment coupling has a jaw, clamp, or threaded member adapted for engaging a tubular or nontubular fixture in a compressive arrangement.

14. The device of claim 3, wherein the second support arm is rigid or deformable and adapted for only engaging the image display.

15. The device of claim 2, wherein the image display further comprises a smartphone, tablet, or handheld display.

16. A method of securing the position of an ultrasound probe and/or ultrasound image display using a holding device to free the hands of the operator for percutaneous ultrasound-guided procedures.

17. The method of claim 16, wherein the positioning is maintained before and/or during an ultrasound-guided procedure or scan.

18. The method of claim 16, wherein a sterile field is maintained for ultrasound-guide procedures comprising a sterile ultrasound probe and/or image display holder device.

19. The device of claim 2, wherein a sterile field for ultrasound-guided procedures is maintained comprising a sterile sleeve for covering the ultrasound probe and/or image display holder.

20. A device for securing the position of an ultrasound probe and ultrasound-guided needle using an ultrasound probe holder with an integrated needle guide to free the hands of the operator for percutaneous ultrasound-guided procedures.

21. The device of claim 20, wherein the integrated needle guide is integrated with the receptacle for the probe.

22. A method of securing the position of an ultrasound probe and ultrasound-guided needle using an ultrasound probe holder with an integrated needle guide to free the hands of the operator for percutaneous ultrasound-guided procedures.

23. The method of claim 22, wherein the position is maintained before or during an ultrasound-guided procedure.

25. The device of claim 2, wherein the device is adapted for securing the position of an ultrasound probe and ultrasound-guided needle using an ultrasound probe holder with an integrated needle guide to free the hands of the operator for percutaneous ultrasound-guided procedures, the ultrasound probe holder adapted to be integrated with a robotic positioning system.

26. The method of claim 17, comprising accurately positioning an ultrasound probe using a probe holder with an optional integrated needle guide to free the hands of the operator for percutaneous ultrasound-guided procedures integrated with a robotic positioning system.
